(19) United States
(12) Patent Application Publication
(10) Pub. No.: US 2026/0108719 A1
(43) Pub. Date: Apr. 23, 2026

(54) NEEDLELESS CONNECTOR

(71) Applicant: CareFusion 303, Inc., San Diego, CA (US)

(72) Inventors: Soon Yong PARK, Cypress, CA (US); Christopher Jesse ZOLLINGER, Chino, CA (US); Jason Andrew WINE, Brea, CA (US); Kuochu Colin HUANG, Walnut, CA (US)

(21) Appl. No.: 19/358,076

(22) Filed: Oct. 14, 2025

Related U.S. Application Data
(60) Provisional application No. 63/709,032, filed on Oct. 18, 2024.

(51) Int. Cl. A61M 39/24; A61M 39/10; A61M 39/00

(52) U.S. Cl. CPC A61M 39/24; A61M 39/10; A61M 2039/0009; A61M 2039/1027; A61M 2039/246; A61M 2039/2493; A61M 2205/0216; A61M 2205/3331

(57) ABSTRACT

Needleless connectors are described that can include a housing and a flexible valve element, the flexible valve element positioned within a cavity of the needleless connector to selectively permit flow between proximal and distal fluid ports of the housing, and the flexible valve element including a valve head, a valve stem, and a valve diaphragm, the flexible valve element further including features to resist obstruction of fluid flow between the valve head and the valve diaphragm, including a fluid flow pathway along any of a shoulder of the valve head and the valve diaphragm, where the fluid flow pathway can be formed by any of a channel and a rib. The flexible valve element can further include a diaphragm formed as a bellows to resist or reduce strain on the flexible valve element when the needleless connector is activated.

### CROSS-REFERENCE TO RELATED APPLICATIONS

This application claims the benefit of U.S. Provisional Application No. 63/709,032, titled “NEEDLELESS CONNECTOR,” filed October 18, 2024, the disclosure of which is incorporated herein by reference in its entirety.

### BACKGROUND

The present disclosure generally relates to connectors, and, in particular, to needleless connectors.

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. Certain needleless connectors may be used in an IV set and may have a self-sealing port to prevent leakage of fluid when a mating medical implement is decoupled from such a needleless connector. Additionally, a needleless connector may include a mechanical valve, for example, a collapsible valve comprising a flexible material for providing the self-sealing port and controlling the flow of fluid within the IV set.

In some applications, needleless connectors may retain or trap fluid within the connector or may have dead spots where a fluid may not move through the connector. In some instances, the needleless connector may not operate as intended due to strain or wear on the collapsible valve.

### SUMMARY

The disclosed subject matter relates to connectors having improved valve elements and housings. In certain embodiments, a needleless connector is disclosed that comprises a housing comprising a cavity, a proximal fluid port in fluid communication with the cavity, and a distal fluid port in fluid communication with the cavity; and a flexible valve element disposed within the cavity to selectively permit flow between the proximal fluid port and the distal fluid port.

In certain embodiments, a needleless connector is disclosed and comprises a housing comprising an inner surface forming a cavity, a proximal fluid port fluidly coupled to the cavity,

a distal fluid port fluidly coupled to the cavity, and a flexible valve element comprising a valve head, a valve stem, and a valve diaphragm, the valve head having a first end, a second end, a shoulder between the first and second ends, the valve stem extending from the valve head in a direction away from the first end of the valve head, and the valve diaphragm between the shoulder and the valve stem, and extending radially outward in a direction away from the second end of the valve head, wherein a fluid flow pathway extends along any of the shoulder and the valve diaphragm, wherein the valve is positioned in the housing with a perimeter of the valve diaphragm coupled to the housing to separate the cavity into a fluid cavity portion and an air cavity portion, and the valve head positioned in the fluid cavity portion and the valve stem positioned in the air cavity portion, and wherein the fluid cavity portion is isolated from the air cavity portion.

In some instances, the present disclosure provides a flexible valve element for a needleless connector, the flexible valve element comprising a valve head having a first end, a second end, a shoulder between the first and second ends, and a neck between the first end and the shoulder, wherein the shoulder comprises a channel that extends in a longitudinal direction between the first end of the valve head and the second end of the valve head, a valve stem that extends from the valve head in a direction away from the first end of the valve head, and a valve diaphragm between the shoulder and the valve stem, the valve diaphragm extending radially outward in a direction away from the second end of the valve head.

In some instances, the present disclosure provides a flexible valve element for a needleless connector, the valve comprising a valve head comprised of a first material, the valve head having a first end, a second end, a shoulder between the first and second ends, and a neck between the first end and the shoulder, a valve stem that extends from the valve head in a direction away from the first end of the valve head, and a valve diaphragm between the shoulder and the valve stem, the valve diaphragm extending radially outward in a direction away from the second end of the valve head, wherein the valve diaphragm is comprised of a second material that is different than the first material.

Accordingly, the present application addresses several operational challenges encountered in prior fluid connections and provides numerous improvements that enable the user to increase safety and efficacy, while more easily and precisely providing needleless connectors with more complete fluid flow therethrough.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a needleless connector in use with an IV set coupled to a patient, in accordance with aspects of the present disclosure.

FIG. 2 is a cross-sectional view of a needleless connector, in accordance with various aspects of the present disclosure.

FIG. 3 is a cross-sectional view of another needleless connector, in accordance with various aspects of the present disclosure.

FIG. 4 is a front perspective view of a flexible valve element, in accordance with various aspects of the present disclosure.

FIG. 5 is a front perspective view of another flexible valve element, in accordance with various aspects of the present disclosure.

FIG. 6 is a partial cross-sectional view of a needleless connector and the flexible valve element of FIG. 5, in accordance with various aspects of the present disclosure.

FIG. 7 is a front perspective view of another flexible valve element, in accordance with various aspects of the present disclosure.

FIG. 8 is a partial cross-sectional view of a needleless connector and the flexible valve element of FIG. 7, in accordance with various aspects of the present disclosure.

### DETAILED DESCRIPTION

The disclosed needleless connector incorporates various features to facilitate the connection, priming, flushing, and/or disconnection of the connector. Features can include, but are not limited to, features of flexible valve elements that can include a valve head, a valve diaphragm, and a valve stem. By providing a needleless connector with one or more of these features, the needleless connector can provide improved connection, priming, flushing, and/or disconnection operation compared to conventional needleless connectors.

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although the following description is directed to the administration of medical fluid to a patient by a medical practitioner using the disclosed needleless connector, it is to be understood that this description is only an example of usage and does not limit the scope of the claims. Various aspects of the disclosed needleless connectors may be used in any application where it is desirable to prevent the transfer of bacteria, microorganisms, and other pathogens while permitting the draw of blood and other fluids from a subject or patient.

The disclosed needleless connector overcomes several challenges discovered with respect to certain conventional connectors. One challenge with certain conventional needleless self-sealing connectors is that certain conventional needleless connectors may cause fluid to become trapped within the needleless connector or portions of a valve therein, may create regions where fluid is resisted from flowing, and thereby reduce the ability to flush or move fluid through the needleless connector. Additionally, another challenge with certain conventional needleless self-sealing connectors is that certain conventional connectors may include a valve element that may become damaged or cease to operate as intended due to strain or wear on the valve element.

Therefore, in accordance with the present disclosure, it is advantageous to provide a needleless connector as described herein that can facilitate connections, priming, flushing, and disconnections. Further, it is advantageous to provide a needleless connector as described herein that can include features that are resilient and readily manufactured.

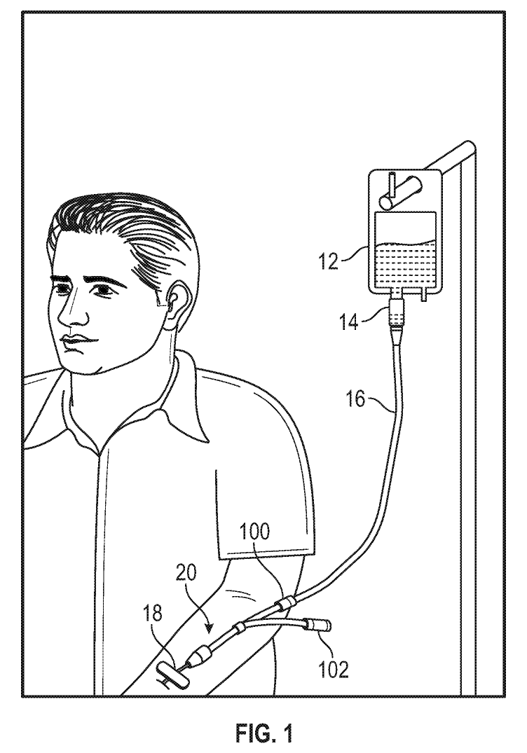

Referring now to the drawings, FIG. 1 illustrates an example of needleless connectors in use in accordance with aspects of the present disclosure. The needleless connectors 100, 102 are shown coupled with a y-site 20, which is being used to direct a fluid to a patient 10. The IV set can include a medicament bag 12, a drip chamber 14, tubing 16, and an IV catheter 18.

A first needleless connector 100 is coupled between a first branch of the y-site 20 and the tubing 16 of the IV set such that the needleless connector 100 is activated and permits a fluid to move from the tubing 16, through the needleless connector 100, and into the IV catheter 18. A second needleless connector 102 is coupled to a second branch of the y-site 20 and is not activated such that the needleless connector 102 resists movement of a fluid into or out of the y-site 20.

When the needleless connector 100, 102 is activated, such as by connection of the needleless connector 100, 102 to another connector, luer fitting, or syringe, a flexible valve element within the needleless connector is biased or compressed to open a fluid pathway through the needleless connector. The flexible valve element is configured to resist the creation of fluid flow dead spots or fluid pockets when the flexible valve element is compressed upon activation of the needleless connector 100, 102. Thus, the flexible valve element is configured to permit a fluid to be flushed or moved through the activated the needleless connector 100, 102 while resisting flow dead spots or pockets where the fluid can become trapped or fluid flow can be obstructed. Additionally, features of the needleless connector in the present disclosure can resist damage, such as tearing or unintended deformation, of the flexible valve element during use of the needleless connector.

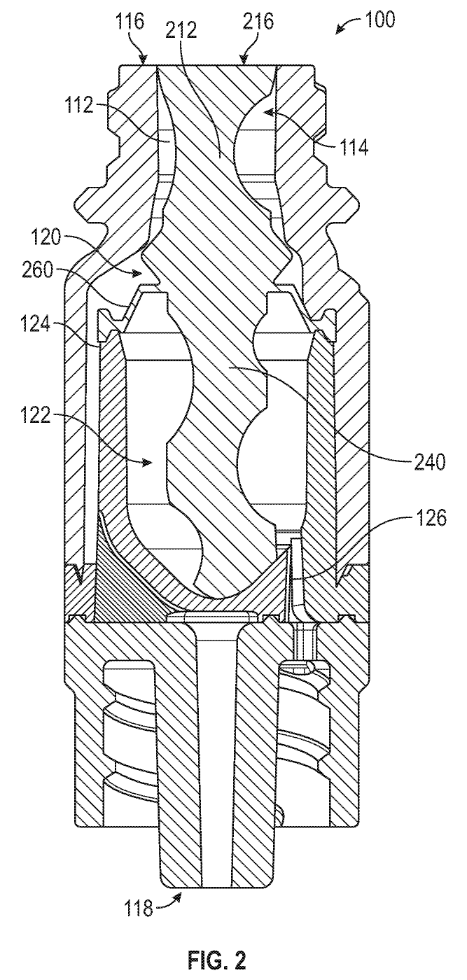

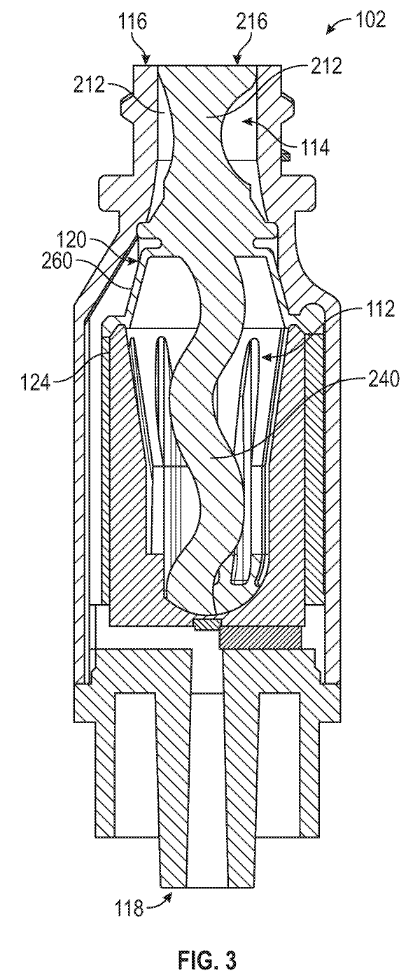

FIGS. 2 and 3 are cross-sectional views of needleless connectors in accordance with various aspects of the present disclosure. FIG. 2 is a needleless connector 100 configured as a positive displacement connector, and FIG. 3 is a needleless connector 102 configured as a negative displacement connector. Each of the needleless connectors include a housing 110 and a flexible valve element 200 positioned within the housing. As shown, the needleless connectors 100, 102 are not activated such that a portion of the flexible valve element 200 obstructs a fluid pathway through the needleless connector to resist movement of a fluid therethrough.

The housing 110 includes a inner surface 112 forming a cavity 114 in the needleless connector which is configured to receive at least a portion of the flexible valve element therein. The cavity 114 is fluidly coupled to fluid ports of the housing 110, including a proximal fluid port 116 and a distal fluid port 118.

As illustrated, fluid flow from an IV set can move toward a patient via a proximal fluid port 116, through a housing 110, through the distal fluid port 118, and to a patient. Further, in some embodiments, blood and other fluids from the patient can be drawn from the distal fluid port 118, through the housing 110, and to the proximal fluid port 116. As shown, the proximal fluid port 116 and the distal fluid port 118 can include any suitable fitting, including, but not limited to Luer fittings. In the depicted example, the proximal fluid port 116 is shown as a female or socket Luer fitting and the distal fluid port 118 is shown as a male or pin Luer fitting.

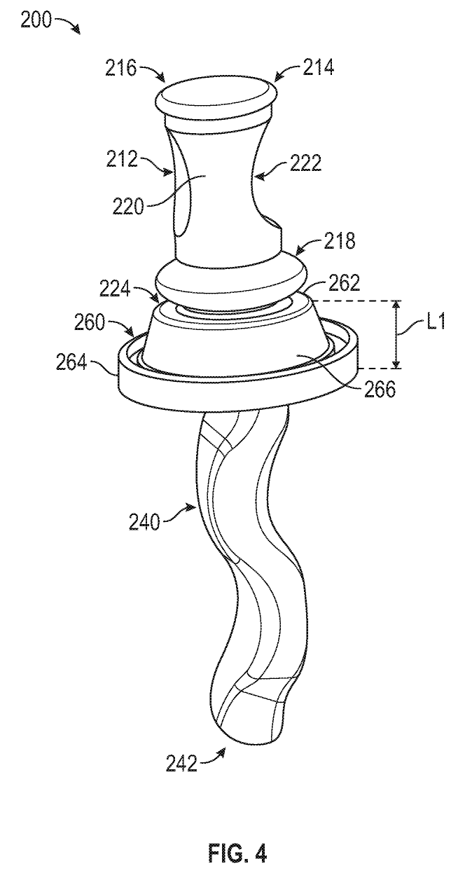

As illustrated in the needleless connectors 100, 102 of FIGS. 2 and 3, and with reference to the flexible valve element illustrated in FIG. 4, the flexible valve element 200 can permit or restrict flow through the proximal fluid port 116 by selectively sealing against the cavity 114 of the housing. The flexible valve element 200 includes a valve head 212 with a sealing surface 214 toward a first end 216 of the flexible valve element, a shoulder 218 between the first end and the second end of the valve head, and a neck 220 that extends between the first end 216 and the shoulder 218 of the valve head.

When the needleless connector is not activated, the flexible valve element 200 is positioned with the valve head 212 at least partially within the proximal fluid port 116 such that the sealing surface and the shoulder 218 of the flexible valve element are engaged against the inner surface 112 of the housing to seal the proximal fluid port 116 and resist movement of a fluid through the proximal fluid port 116.

The valve head 212 can further include one or more cut portion 222 to facilitate flow past the flexible valve element 200 by readily deforming along the one or more cut portion 222 upon insertion of a mating medical implement into the needleless connector.

The flexible valve element 200 also includes a valve stem 240 that extends from a second end 224 of the valve head in a direction away from the first end 216 of the valve head. When the flexible valve element 200 is positioned within the needleless connector, a distal end 242 of the valve stem is engaged against the housing 110 such that the valve head is positioned in the proximal fluid port 116. Further, the valve stem 240 resists movement of the valve head 212 in a direction toward the distal fluid port 118.

The flexible valve element 200 further includes a diaphragm 260 that is positioned proximal to the second end 224 of the valve head and extends radially outward in a direction away from the second end 224 of the valve head. The valve diaphragm 260 includes a proximal end 262

and a distal end 264. The proximal end 262 of the valve diaphragm is coupled with the valve head such that the valve diaphragm extends radially outward from the valve head to the distal end 264 of the valve diaphragm. The proximal end 262 of the valve diaphragm is positioned between the second end 224 of the valve head and the valve stem 240. In some embodiments, it is contemplated that the proximal end 262 of the valve diaphragm can be located between the shoulder of the valve head and the second end 224 of the valve head.

When the valve diaphragm 260 is in a neutral or unstretched position, the valve diaphragm 260 has a length L1 from the proximal end 262 of the valve diaphragm to a distal end 264 of the valve diaphragm. When the valve diaphragm is stretched in a longitudinal direction, between the proximal and distal ends 262, 264, the valve diaphragm 260 can have a length L2 that is longer than the length L1.

The distal end 264 of the valve diaphragm forms a circumference or perimeter that extends around the outer surface of the valve head and can be coupled with a portion of the housing 110. The valve diaphragm 260 has a cone shape with a cross-sectional width of the valve diaphragm increasing in a direction from the proximal end 262 toward the distal end 264 of the valve diaphragm. Although the valve diaphragm 260 is shown extending from the proximal end 262 to the distal end 264 in a direction away from the first end 216 of the valve head and along the valve stem 240, it is contemplated that the valve diaphragm could be configured to extend radially outward to form a planar shape or can extend radially outward in a direction toward the first end 216 of the valve head.

The valve diaphragm 260 is configured to couple with a portion of the housing to separate the cavity 114 into a fluid cavity portion 120 and an air cavity portion 122, where the fluid cavity portion 120 is fluidly isolated from the air cavity portion 122. To separate the cavity 114 into a fluid cavity portion 120 and an air cavity portion 122, the distal end 264 of the valve diaphragm is coupled to a wall 124 within the housing that forms a valve seat for the flexible valve element.

The air cavity portion 122 is formed or bounded by an inner surface of the wall 124, a lower surface of the valve diaphragm 260, and an outer surface of the valve stem 240. In some embodiments of the present disclosure in which the housing 110 includes an air passage 126 that extends from the air cavity portion 122 to an ambient atmosphere adjacent to an outer surface of the housing, the needleless connector 100 functions as a positive displacement device (FIGURE2).

In embodiments of the present disclosure in which the air cavity portion 122 is isolated from an ambient atmosphere adjacent to the outer surface of the housing, the needleless connector 102 functions as a negative displacement device FIG. 3.

The fluid cavity portion 120 is formed or bounded by the inner surface 112 of the housing, an outer surface an outer surface of the wall 124, an outer surface of the valve head 212, and an upper surface 266 of the valve diaphragm. The fluid cavity portion 120 also forms at least a portion of a fluid pathway that extends form the proximal fluid port 116 to the distal fluid port 118 when the needleless connector is activated.

During operation of the needleless connector, a male fitting of a medical implement can be introduced into the proximal fluid port 116 to activate the needleless connector. Upon introduction of the male fitting into the proximal fluid port 116, the flexible valve element 200 can be sufficiently elastic to deform or bend out of sealing engagement with the inner surface 112 of the housing to permit fluid flow between the needleless connector and the medical implement. When the needleless connector is activated a fluid can flow along the fluid pathway of the fluid cavity portion 120 and between the proximal fluid port 116 and the distal fluid port 118.

Upon disconnection of the male fitting from the proximal fluid port 116, the flexible valve element 200 can return to its original shape such that the valve head 212 extends into the proximal fluid port 116 such that the needleless connector is not activated. When the needleless connector is not activated, the fluid cavity portion 120 remains isolated from the air cavity portion 122 and the proximal fluid port 116.

In the depicted example of the positive displacement device in FIG. 2, the volume of the air cavity portion 122 decreases such that a fluid is drawn in from the proximal fluid port 116 or the distal fluid port 118 when a male fitting is inserted into the proximal fluid port 116. When the male fitting is removed from the proximal fluid port 116, fluid is expelled from the fluid cavity portion 120, thereby flushing the needleless connector 100.

The flexible valve element 200 can be formed from silicone or other elastomeric materials to resiliently deform when the needleless connector is activated and reform to allow sealing of the proximal fluid port 116 when the needleless connector is not activated. When the needleless connector is activated, any of the valve head 212 and the valve stem 240 can compress, bend, fold, or twist such that the valve head 212 moves in a longitudinal direction away from the proximal fluid port 116 and closer to the distal fluid port 118. As the valve head 212 moves toward the distal fluid port 118, the valve diaphragm 260 stretches in a radial direction such that the valve diaphragm 260 has a length L2 which is greater than the length L1.

The flexible valve element 200 can be formed of more than material or can be formed of one material having different characteristics in different portions of the flexible valve element. In some embodiments of the present disclosure, the valve head 212 can comprise a first durometer hardness rating, and the valve diaphragm 260 can comprise a second durometer hardness rating, where the second durometer hardness rating is less than the first durometer hardness rating. In one example, the valve head 212 is formed of a first material having a first durometer hardness rating, and the valve diaphragm 260 is formed of second material having a second durometer hardness rating, where the first material is different than the second material, and the first and second durometer hardness ratings are different. In another example, the different first and second durometer hardness ratings can be achieved by forming the valve diaphragm 260 with a thickness that is less than a thickness of the valve head 212.

The flexible valve element 200 can be formed as a unitary component with the valve head 212, the valve stem 240, and the valve diaphragm 260 formed as a unitary and single component. In some instances, one or more portion of the flexible valve element 200 can be separately formed and joined with other portions of the flexible valve element 200 during manufacturing and/or using subsequent processing steps. For example, the valve diaphragm 260 can be overmolded or otherwise coupled to any of the valve head 212 and the valve stem 240.

In some embodiments of the present disclosure, a portion of the flexible valve element can include a structure or features that provides a fluid flow pathway along a surface of the flexible valve element. The flexible valve element can be configured so that the fluid flow pathway resists or reduces the instance of fluid becoming trapped or obstructed in or around the flexible valve element when the needleless connector is activated.

The fluid flow pathway can be formed by a ridge and/or channel extending along any of the upper surface 266 of the valve diaphragm and/or the valve head 212. The ridge can be shaped as a convex surface, such as a rib, and the channel can be formed as a concave surface, such as a groove. In any instance, the fluid flow pathway is configured to permit fluid to move between the upper surface 266 of the valve diaphragm and the valve head 212. When the needleless connector is activated, the valve head 212 can compress or fold toward the valve diaphragm 260. Further, the fluid flow pathway can permit fluid to move along a pocket or regions in which a fluid can otherwise become trapped when the upper surface 266 of the valve diaphragm engaged against the valve head 212, thereby ensuring and increasing the reliability of flushing or moving fluid through the needleless connector.

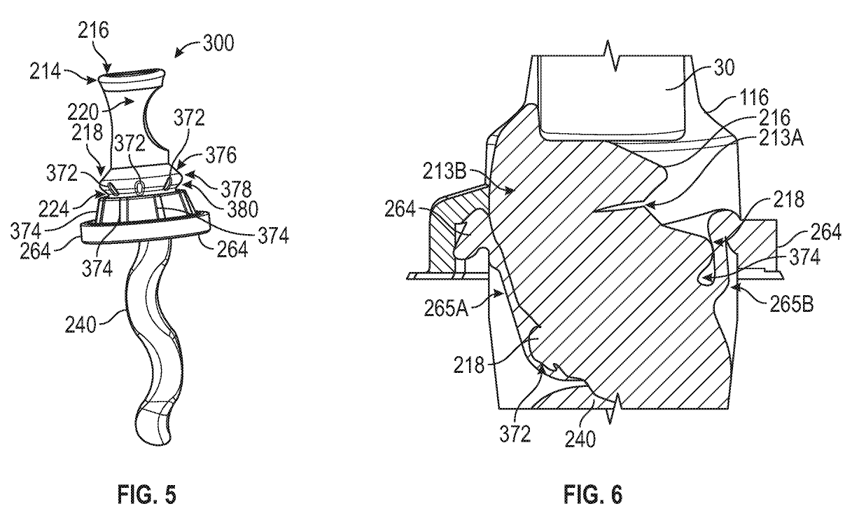

FIG. 5 illustrates an exemplary embodiment of a flexible valve element 300 with channel 372 extending along the valve head and a rib 374 extending along the valve diaphragm. The flexible valve element 300 includes features that are similar to the flexible valve element 200 shown in FIGS. 2-4. Accordingly, certain features of the flexible valve element 300 are referred to with similar reference numerals used in reference to the flexible valve element 200.

The channel 372 extends along at least a portion of the shoulder 218 in a longitudinal direction between the first end 216 of the valve head and the second end 224 of the valve head. The shoulder comprises a top surface 376 facing in a direction toward the neck 220 of the valve head, and a bottom surface 380 facing in a direction toward the valve diaphragm 260. The top surface 376 of the shoulder extends radially outward in a direction away from the neck 220 to an apex 378 of the shoulder. The bottom surface 380 of the shoulder extends radially outward in a direction away from the second end 224 of the valve head to the apex 278.

In some embodiments of the present disclosure, such as the example shown in FIG. 5, the channel 372 extends along the bottom surface 380 of the shoulder 218, in a longitudinal direction between the second end 224 of the valve and the apex 378 of the shoulder. The channel 372 can terminate before the apex 378 of the shoulder or can extend through the apex 378 of the shoulder. In some embodiments of the present disclosure, the channel 372 extends only along the bottom surface 380 and the apex 378 of the shoulder.

The rib 374 extends along the upper surface 266 of the valve diaphragm in a longitudinal direction between the valve head 212 and the distal end 264 of the valve diaphragm. The valve diaphragm 260 includes a plurality of ribs 374 that are radially spaced apart around a circumferent of the valve diaphragm 260. The plurality of ribs 374 are spaced apart from each other with a spacing between adjacent ribs 374 that is equal; however, the present disclosure contemplates embodiments in which spacing between adjacent ribs 374 is different or not equal.

Additionally, the plurality of ribs 374 can positioned relative to a plurality of channels 372 on a circumferent of the shoulder 218. In some embodiments of the present disclosure, the plurality of ribs 374 on the valve diaphragm are radially offset from the plurality of channels 372 on the shoulder. In some embodiments, the plurality of ribs 374 on the valve diaphragm are radially aligned with the plurality of channels 372 on the shoulder.

The present disclosure also contemplates embodiments in which the flexible valve element comprises only one of the features of a channel 372 or a rib 374, and embodiments in which the flexible valve element comprises a combination of a channel 372 and a rib 374. For example, the flexible valve element can include one or more channel 372 extending along any of the shoulder 218 and/or the valve diaphragm 260, and no rib 374. In another example, the flexible valve element can include one or more rib 374 extending along any of the shoulder 218 and/or the valve diaphragm 260, and no channel 372. In yet another example, the flexible valve element can include one or more channel 372 and one or more rib 374 extending along any of the shoulder 218 and/or the valve diaphragm 260.

In another example embodiment of the present disclosure, the channel 372 and the rib 374 can be formed by another shape that can permit movement of a fluid relative to the flexible valve element. For example, the channel 372 can be formed as a concave depression or dimple extending into the surface of the shoulder 218 and/or the valve diaphragm 260. In another example the rib 374 can be formed as a convex surface or bump extending away from the surface of the shoulder 218 and/or the valve diaphragm 260.

Referring to FIG. 6, a portion of the flexible valve element 300 is shown when the needleless connector is activated by a male fitting 30. The valve head 212 is axially compressed in a direction toward the valve stem 240. A first circumferential portion 213A of the valve head is folded along the neck 220 and a second circumferential portion 213B of the valve head is stretched. Additionally, a first portion 265A of the valve diaphragm is longitudinally stretched and a second portion 265B of the valve diaphragm is folded. Each of the first portion 265A and the second portion 265B of the valve diaphragm are engaged against the shoulder 218 of the valve head. Although the region in which the valve diaphragm 260 is engaged against the shoulder 218 could form a dead spot or pocket where fluid flow is obstructed, the fluid flow pathway formed by the channel 372 and the rib 374 permit fluid to move between the valve diaphragm 260 and the valve head 212, thereby increasing the ability and efficiency of moving a fluid through the needleless connector.

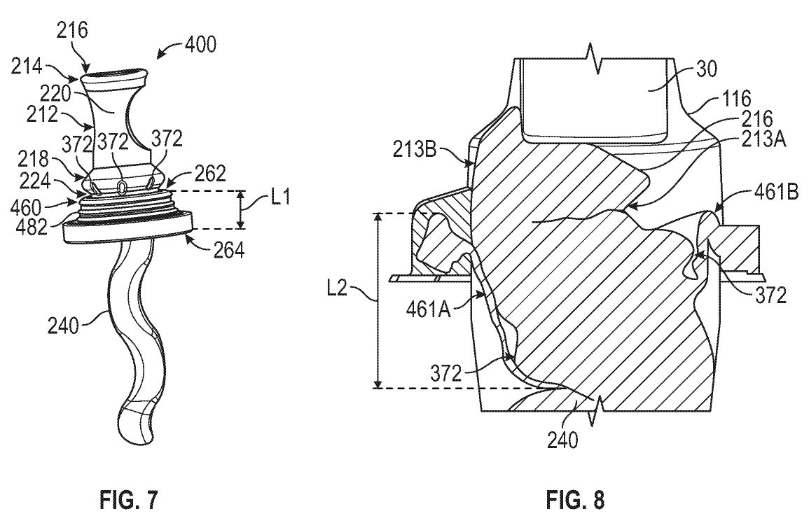

In some embodiments of the present disclosure, the valve diaphragm can be configured to resist strain when the needleless connector is activated, thereby resisting potential damage and unintended performance of the flexible valve element. To resist stain when the needleless connector is activated, embodiments of a flexible valve element 400 can include a valve diaphragm having a shape of a bellows 482, as shown in FIG. 7. The flexible valve element 400 includes features that are similar to the flexible valve element 200 shown in FIGS. 2–4 and the flexible valve element 300 shown in FIGS. 5 and 6. Accordingly, certain features of the flexible valve element 400 are referred to with similar reference numerals used in reference to the flexible valve elements 200, 300.

The bellows 482 shaped portion of the valve diaphragm extends between the valve head 212 and the distal end 264 at the perimeter of the valve diaphragm 460. The bellows 482 is shaped by a corrugated cross-section of the valve diaphragm forming concentric rings along at least the upper surface 266 of the valve diaphragm. When the valve diaphragm 460 is in a neutral or unstretched position, the valve diaphragm 460 has a length L1 from a proximal end 262 of the valve diaphragm to a distal end 264 of the valve diaphragm. When the valve diaphragm 460 is biased, such as when the needleless connector is activated, a portion of the valve diaphragm 460 can be stretched and another portion of the valve diaphragm 460 can be compressed or folded. In some embodiments of the present disclosure, when the needleless connector is activated, at least a portion of the valve diaphragm 460 along the bellows 482 is stretched from length L1 to length L2, where the length L2 is greater than the length L1. In some embodiments of the present disclosure, the length L2 can be 175% greater than the length L1.

Referring to FIG. 8, a first portion of the flexible valve element 400 is shown when the needleless connector is activated by a male fitting 30. A first portion 461A of the valve diaphragm is longitudinally stretched and a second portion 461B of the valve diaphragm is folded. The first portion 461A of the valve diaphragm that is longitudinally stretched can have the length L2, and the second portion 461B of the valve diaphragm that is folded can have the length L1.

Each of the first portion 461A and the second portion 461B of the valve diaphragm are engaged against the shoulder 218 of the valve head. Although the region in which the valve diaphragm 460 is engaged against the shoulder 218 could form a dead spot or pocket where fluid flow is obstructed, the fluid flow pathway formed by channels 372 along the shoulder permit fluid to move between the valve diaphragm 460 and the valve head 212. Thus, the flexible valve element 400 can provide permit fluid to move through the needleless connector and resist strain which could otherwise result in damage and unintended performance of the flexible valve element.

Various examples of aspects of the subject technology are described below for convenience. These are provided as examples and do not limit the subject technology. Further, the examples may be combined in any combination, and placed into respective embodiments.

A needleless connector comprising a housing comprising: an inner surface forming a cavity, a proximal fluid port fluidly coupled to the cavity, a distal fluid port fluidly coupled to the cavity; and a flexible valve element comprising a valve head, a valve stem, and a valve diaphragm, the valve head having a first end, a second end, a shoulder between the first and second ends, the valve stem extending from the valve head in a direction away from the first end of the valve head, and the valve diaphragm between the shoulder and the valve stem, and extending radially outward in a direction away from the second end of the valve head, wherein a fluid flow pathway extends along any of the shoulder and the valve diaphragm; wherein the valve is positioned in the housing with a perimeter of the valve diaphragm coupled to the housing to separate the cavity into a fluid cavity portion and an air cavity portion, and the valve head positioned in the fluid cavity portion and the valve stem positioned in the air cavity portion, and wherein the fluid cavity portion is isolated from the air cavity portion.

Wherein shoulder comprises a channel that extends in a longitudinal direction between the first end of the valve head and the second end of the valve head, and wherein the fluid flow pathway is formed by the channel.

Wherein the valve head comprises a neck between the first end and the shoulder, and the shoulder comprises a top surface and a bottom surface, the top surface extends radially outward in a direction away from the neck to an apex, and the bottom surface extends radially outward in a direction away from the second end of the valve head to the apex.

Wherein the channel extends along the bottom surface of the shoulder.

Wherein the valve diaphragm comprises more than one rib extending along the valve diaphragm and the fluid flow pathway is formed by a recessed surface between adjacent ribs of the more than one rib.

Wherein the valve diaphragm comprises a bellows shape between the valve head and the perimeter of the valve diaphragm.

Wherein the valve head comprises a first material, and the valve diaphragm comprises a second material that is different than the first material.

Wherein a material of the valve head comprises a first durometer hardness rating, and the valve diaphragm comprises a second durometer hardness rating, and wherein the second durometer hardness rating is less than the first durometer hardness rating.

Wherein the valve stem comprises a plurality of bends.

A flexible valve element for a needleless connector, the valve comprising: a valve head having a first end, a second end, a shoulder between the first and second ends, and a neck between the first end and the shoulder, wherein the shoulder comprises a channel that extends in a longitudinal direction between the first end of the valve head and the second end of the valve head; a valve stem that extends from the valve head in a direction away from the first end of the valve head; and a valve diaphragm between the shoulder and the valve stem, the valve diaphragm extending radially outward in a direction away from the second end of the valve head.

Wherein the shoulder comprises a top surface and a bottom surface, the top surface extends radially outward in a direction away from the neck to an apex, and the bottom surface extends radially outward in a direction away from the second end of the valve head to the apex, and wherein the channel extends along the bottom surface of the shoulder.

Wherein the shoulder comprises a top surface and a bottom surface, the top surface extends radially outward in a direction away from the neck to an apex, and the bottom surface extends radially outward in a direction away from the second end of the valve head to the apex, and wherein the channel extends along the bottom surface of the shoulder.

Wherein the channel extends through the apex of the shoulder.

Wherein the channel does not extend along the top surface of the shoulder.

Wherein the valve diaphragm comprises a perimeter, a top surface, and a rib, the top surface facing in a direction toward the first end of the valve head and extending from the valve head to the perimeter of the valve diaphragm, and the rib extending longitudinally in a direction between the valve head and the perimeter of the valve diaphragm.

Wherein the valve diaphragm comprises more than one rib extending longitudinally along a top surface of the valve diaphragm and in a direction between the valve head and a perimeter of the valve diaphragm.

Wherein the valve diaphragm comprises a bellows shape between the valve head and a perimeter of the valve diaphragm.

Wherein the valve head comprises a first material, and the valve diaphragm comprises a second material that is different than the first material.

A flexible valve element for a needleless connector, the valve comprising: a valve head comprised of a first material, the valve head having a first end, a second end, a shoulder between the first and second ends, and a neck between the first end and the shoulder; a valve stem that extends from the valve head in a direction away from the first end of the valve head; and a valve diaphragm between the shoulder and the valve stem, the valve diaphragm extending radially outward in a direction away from the second end of the valve head, wherein the valve diaphragm is comprised of a second material that is different than the first material.

Wherein the first material comprises a first durometer hardness rating, and the second material comprises a second durometer hardness rating, and wherein the second durometer hardness rating is less than the first durometer hardness rating.

Wherein the valve diaphragm is over-molded to the valve head.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. A needleless connector comprising:
a housing comprising an inner surface forming a cavity, a proximal fluid port fluidly coupled to the cavity, a distal fluid port fluidly coupled to the cavity; and
a flexible valve element comprising a valve head, a valve stem, and a valve diaphragm, the valve head having a first end, a second end, a shoulder between the first and second ends, the valve stem extending from the valve head in a first direction away from the first end of the valve head, and the valve diaphragm between the shoulder and the valve stem, and extending radially outward in a second direction away from the second end of the valve head, wherein a fluid flow pathway extends along any of the shoulder and the valve diaphragm;
wherein the flexible valve element is positioned in the housing with a perimeter of the valve diaphragm coupled to the housing to separate the cavity into a fluid cavity portion and an air cavity portion, and the valve head positioned in the fluid cavity portion and the valve stem positioned in the air cavity portion, and wherein the fluid cavity portion is isolated from the air cavity portion.

2. The needleless connector of claim 1, wherein the shoulder comprises a channel that extends in a longitudinal direction between the first end of the valve head and the second end of the valve head, and wherein the fluid flow pathway is formed by the channel.

3. The needleless connector of claim 2, wherein the valve head comprises a neck between the first end and the shoulder, and the shoulder comprises a top surface and a bottom surface, the top surface extends radially outward away from the neck to an apex, and the bottom surface extends radially outward away from the second end of the valve head to the apex.

4. The needleless connector of claim 3, wherein the channel extends along the bottom surface of the shoulder.

5. The needleless connector of claim 1, wherein the valve diaphragm comprises more than one rib extending along the valve diaphragm and the fluid flow pathway is formed by a recessed surface between adjacent ribs of the more than one rib.

6. The needleless connector of claim 1, wherein the valve diaphragm comprises a bellows shape between the valve head and the perimeter of the valve diaphragm.

7. The needleless connector of claim 1, wherein the valve head comprises a first material, and the valve diaphragm comprises a second material that is different than the first material.

8. The needleless connector of claim 1, wherein a material of the valve head comprises a first durometer hardness rating, and the valve diaphragm comprises a second durometer hardness rating, and wherein the second durometer hardness rating is less than the first durometer hardness rating.

9. The needleless connector of claim 1, wherein the valve stem comprises a plurality of bends.

10. A flexible valve element for a needleless connector, the flexible valve element comprising:
a valve head having a first end, a second end, a shoulder between the first and second ends, and a neck between the first end and the shoulder, wherein the shoulder comprises a channel that extends in a longitudinal direction between the first end of the valve head and the second end of the valve head;
a valve stem that extends from the valve head in a first direction away from the first end of the valve head; and
a valve diaphragm between the shoulder and the valve stem, the valve diaphragm extending radially outward in a second direction away from the second end of the valve head.

11. The flexible valve element of claim 10, wherein the shoulder comprises a top surface and a bottom surface, the top surface extends radially outward in a direction away from the neck to an apex, and the bottom surface extends radially outward away from the second end of the valve head to the apex, and wherein the channel extends along the bottom surface of the shoulder.

12. The flexible valve element of claim 11, wherein the channel extends through the apex of the shoulder.

13. The flexible valve element of claim 11, wherein the channel does not extend along the top surface of the shoulder.

14. The flexible valve element of claim 10, wherein the valve diaphragm comprises a perimeter, a top surface, and a rib, the top surface facing toward the first end of the valve head and extending from the valve head to the perimeter of the valve diaphragm, and the rib extending longitudinally between the valve head and the perimeter of the valve diaphragm.

15. The flexible valve element of claim 10, wherein the valve diaphragm comprises more than one rib extending longitudinally along a top surface of the valve diaphragm and in a direction between the valve head and a perimeter of the valve diaphragm.

16. The flexible valve element of claim 10, wherein the valve diaphragm comprises a bellows shape between the valve head and a perimeter of the valve diaphragm.

17. The flexible valve element of claim 10, wherein the valve head comprises a first material, and the valve diaphragm comprises a second material that is different than the first material.

18. A flexible valve element for a needleless connector, the flexible valve element comprising:
a valve head comprised of a first material, the valve head having a first end, a second end, a shoulder between the first and second ends, and a neck between the first end and the shoulder;
a valve stem that extends from the valve head in a first direction away from the first end of the valve head; and
a valve diaphragm between the shoulder and the valve stem, the valve diaphragm extending radially outward in a second direction away from the second end of the valve head, wherein the valve diaphragm is comprised of a second material that is different than the first material.

19. The flexible valve element of claim 18, wherein the first material comprises a first durometer hardness rating, and the second material comprises a second durometer hardness rating, and wherein the second durometer hardness rating is less than the first durometer hardness rating.

20. The flexible valve element of claim 18, wherein the valve diaphragm is over-molded to the valve head.
